SEQUENCE LISTING (1 sequence)

SEQ ID NO: 1 - protein, 20 aa, synthetic construct
AFCGGRCRGFRRRLFCTKAC

(19) United States
(12) Patent Application Publication
(10) Pub. No.: US 2025/0289850 A1
(43) Pub. Date: Sep. 18, 2025

(54) NOVEL ANTIMICROBIAL PEPTIDES AND METHODS OF USE THEREOF

(71) Applicant: QATAR UNIVERSITY, Doha (QA)

(72) Inventors: Sergio CROVELLA, Doha (QA); Nahla Omer Ahmed ELTAI, Doha (QA); Nuura Adam Mohamed ABDI, Doha (QA)

(21) Appl. No.: 19/079,689

(22) Filed: Mar. 14, 2025

Related U.S. Application Data
(60) Provisional application No. 63/565,734, filed on Mar. 15, 2024.

(51) Int. Cl. C07K 7/08; A61K 38/00; A61P 31/04

(52) U.S. Cl. CPC C07K 7/08; A61P 31/04; A61K 38/00

(57) ABSTRACT

A composition comprising a peptide comprising a sequence that has at least 85% identity with the amino acid sequence set forth in SEQ ID NO:1. Also provided is a method of treating or preventing a disease comprising administering a pharmaceutical composition comprising an therapeutically effective amount of the peptide.

### CROSS-REFERENCE TO RELATED APPLICATIONS

This application claims the benefit of U.S. Provisional Application No. 63/565,734, filed Mar. 15, 2024. The entire contents of the above-identified application are hereby fully incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 11, 2025, is named “432743.10401.xml” and is 1,786 bytes in size. The sequence listing contained in this .XML file is part of the specification and is hereby incorporated by reference hercin in its entirety.

### TECHNICAL FIELD

The subject matter disclosed herein is generally directed to compositions comprising antimicrobial peptides and methods of treating diseases using the compositions.

### BACKGROUND

Antibiotic resistance is a paramount global health issue, with numerous bacterial strains continually fortifying their resistance against diverse antibiotics. This surge in resistance levels primarily stems from the overuse and misuse of antibiotics in human, animal, and environmental contexts. The World Health Organization (WHO) has underscored the alarming case with which pathogens are developing antibiotic resistance, portraying a striking and persistent trait among bacteria.

Among the array of suggested molecules, natural antimicrobial peptides (AMPs) are gaining substantial recognition in research. Natural AMPs are diminutive, biologically active proteins present in both prokaryotes and eukaryotes. Natural AMPs have been used against various pathogens, including bacteria, fungi, and viruses. However, currently available AMPs have drawbacks such as a short half-life, susceptibility to protease degradation, and cytotoxicity to host cells.

Therefore, there is a critical need to develop new molecules endowed with antibacterial activity to supplant currently employed antibiotics.

### SUMMARY

In one aspect, the present disclosure provides a composition comprising a peptide comprising a sequence that has at least 85% identity with the amino acid sequence set forth in SEQ ID NO:1. In some embodiments, the peptide has at least 90% identity with the amino acid sequence set forth in SEQ ID NO:1. In some embodiments, the peptide has at least 95% identity with the amino acid sequence set forth in SEQ ID NO:1. In some embodiments, the peptide comprises the amino acid sequence set forth in SEQ ID NO:1.

In another aspect, the present disclosure provides a pharmaceutical composition comprising a therapeutically effective amount of the peptide of claim 1 and a pharmaceutically acceptable carrier. In some embodiments, the peptide is formulated in a nanoparticle. In some embodiments, the nanoparticle comprises nanofibers. In some embodiments, the nanofibers comprise graphene nanofibers.

In another aspect, the present disclosure provides a method of treating or preventing a disease comprising administering the pharmaceutical composition of claim 5 to a subject in need thereof. In some embodiments, the disease is a bacterial infection. In some embodiments, the bacterial infection is an infection by a gram-positive bacterium. In some embodiments, the gram-positive bacterium is Staphylococcus aureus. In some embodiments, the Staphylococcus aureus is Methicillin-Resistant Staphylococcus aureus. In some embodiments, the bacterial infection is an infection by a gram-negative bacterium. In some embodiments, the gram-negative bacterium is Escherichia coli.

In some embodiments, the disease is skin infection. In some embodiments, the disease is an auto-inflammatory skin disease. In some embodiments, the auto-inflammatory skin disease is hidradenitis suppurativa. In some embodiments, the disease is cancer.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1B. Antibacterial effect of the different concentrations of the AMP on MSRA. FIG. 1A. Image of the 96-well plate with the MRSA treated with different AMP concentrations (2000, 1000, 500, 250, 125, 62.5, 31.25, 15.6, 7.8 μg/ml) in wells 2, 3, 4, 5, 6, 7, 8, 9, 10 respectively. Rows B, C and D represent the N=3 Replicates for each concentration. Turbidity of the treated wells was compared to the MRSA control growth (Row F). FIG. 1B. MRSA was treated with the following AMP concentrations 2000, 1000, 500, 250 μg/ml, where 500 μg/ml was determined as the MIC, with concentrations higher than 250 μg/ml not affecting MRSA growth. Data are mean±SEM for n=3. Statistical analysis was performed using one-way ANOVA followed by Bonferroni Post-tests (*p<0.05).

FIGS. 2A-2B. Antibacterial effect of the different concentrations of the AMP on E. coli. FIG. 2A. Image of the 96-well plate with the E. coli treated with different AMP concentrations (2000, 1000, 500, 250, 125, 62.5, 31.25, 15.6, 7.8 μg/ml) in wells 2, 3, 4, 5, 6, 7, 8, 9, 10 respectively. Rows B, C and D represent the N=3 Replicates for each concentration. Turbidity of the treated wells was compared to the E. coli control growth (Row F). FIG. 2B. E. coli was treated with the following AMP concentrations 2000, 1000, 500, 250 μg/ml, where 500 μg/ml was determined as the MIC, with concentrations higher than 250 μg/ml did not affect E. coli growth. Data are mean±SEM for n=3. Statistical analysis was performed using one-way ANOVA followed by Bonferroni Post-tests (*p<0.05).

FIG. 3A. CFU count of MRSA biofilms. FIG. 3B. CFU count of E. coli biofilms.

FIGS. 4A-4D. The effect of different concentrations of the synthetic AMP on the MRSA biofilm. FIG. 4A. Image of the 96-well plate with the MRSA biofilms treated with different AMP concentrations. FIG. 4B. Close-up image of a dried bead of the MRSA biofilm treated with 2000 μg/mL treatment and a dried bead of the MRSA biofilm control. FIG. 4C. SEM image of a glass head surface of the MRSA biofilm control. FIG. 4D. SEM image of a glass bead surface of the MRSA biofilm treated with 2000 μg/mL for 24 hours at magnification 10.000×.

FIGS. 5A-5D. The effect of different concentrations of the synthetic AMP on the E. coli biofilm. FIG. 5A. Image of the 96-well plate with the E. coli biofilms treated with different AMP concentrations. FIG. 5B. Close-up image of a dried bead of the E. coli biofilm treated with 2000 μg/mL treatment and a dried bead of the E. coli biofilm control. FIG. 5C. SEM image of a glass bead surface of the E. coli biofilm control. FIG. 5D. SEM image of a glass bead surface of the E. coli biofilm treated with 2000 μg/mL for 24 hours at magnification 10,000×.

FIGS. 6A-6B. Effect of the AMP on the fibroblast viability (FIG. 6A) and cytotoxicity (FIG. 6B). Data are mean±S.E.M for n=4: Statistical analysis for effect between different groups was analyzed by two-way ANOVA followed by Bonferroni Posttests. Statistical significance was noted at *p<0.05.

FIGS. 7A-7B. The genotoxicity effect of the AMP on the fibroblast compared to the positive control where cells were treated with H₂O₂. FIG. 7A. Representative fluorescent images showing the comet tail of fragmented DNA and extensive DNA migration caused by the +Ve, while treatments with the AMP concentrations 2000, 500 μg/ml did not cause any comet tail and showed no DNA migration. Images were taken using OLYMPUS BX62, at magnification 40×, attached to an OLYMPUS DP73 digital camera, image analysis was done using ImageJ application (OpenComet plugin). FIG. 7B. Quantification of the extent of DNA migration using tail moments to compare the effect of the different AMP concentration with the +Ve and −Ve. Data are mean±S.E.M for n=4. Statistical analysis for effect between different groups was done by one-way ANOVA followed by Bonferroni Posttests. Statistical significance was noted at *p<0.05.

The figures herein are for illustrative purposes only and are not necessarily drawn to scale.

### DETAILED DESCRIPTION

### Definitions

As used herein, the singular forms “a”, “an”, and “the” include both singular and plural referents unless the context clearly dictates otherwise.

The terms “subject,” “individual,” and “patient” are used interchangeably herein to refer to a vertebrate, e.g., a mammal, such as a human. Mammals include, but are not limited to, simians, humans, pigs, cats, cattle, deer, dogs, ferrets, gaurs, goats, horses, mouses, mouflons, mules, rabbits, rats, sheep, and primates. Tissues, cells and their progeny of a biological entity obtained in vivo or cultured in vitro are also encompassed.

The term “pharmaceutical composition” refers to a composition that comprises an AMP disclosed herein and one or more pharmaceutically acceptable carriers that is conventional in the art and that is suitable for administration to cells or to a subject. The term “pharmaceutically acceptable” as used throughout this specification is consistent with the art and means compatible with the other ingredients of a pharmaceutical composition and not deleterious to the recipient thereof. A pharmaceutical composition is a preparation that is in such form as to permit the biological activity of the active ingredient to be effective, and that contain no additional components that are unacceptably toxic to an individual to which the formulation or composition would be administered. Such compositions may be sterile.

The term “carrier” or “excipient” includes any and all solvents, diluents, buffers (e.g., neutral buffered saline or phosphate buffered saline), solubilizes, colloids, dispersion media, vehicles, fillers, chelating agents (e.g., EDTA or glutathione), amino acids (c.g., glycine), proteins, disintegrants, binders, lubricants, wetting agents, emulsifiers, sweeteners, colorants, flavorings, aromatizes, thickeners, agents for achieving a depot effect, coatings, antifungal agents, preservatives, stabilizers, antioxidants, tonicity controlling agents, absorption delaying agents, and the like. The use of such media and agents for pharmaceutical active components is well known in the art. Such materials should be non-toxic and should not interfere with the activity of the cells or active components.

The term “therapeutically effective amount” refers to an amount (e.g., of an AMP) that is sufficient to provide a therapeutic benefit to a patient in the treatment or management of a disease or disorder, or to delay or minimize one or more symptoms associated with the disease or disorder.

The term “treating” or “treatment” of any disease or disorder refers, in certain embodiments, to ameliorating a disease or disorder, either physically (c.g., stabilization of a discernable symptom) or physiologically (e.g., stabilization of a physical parameter) or both. In yet another embodiment, “treating” or “treatment” of a disease refer to executing a protocol, which may include administering one or more therapeutic agents to an individual (human or otherwise), in an effort to obtain beneficial or desired results in an individual, including clinical and cosmetic results. Beneficial or desired clinical results include, but are not limited to, alleviation or amelioration of one or more symptoms, diminishment of extent of disease(s), stabilized (i.e., not worsening) state of disease, preventing spread or increase in severity of disease, delay or slowing of disease progression, amelioration or palliation of the disease state, and remission (whether partial or total). “Treatment” can also mean prolonging survival as compared to expected survival of an individual not receiving treatment. Further, “treating” and “treatment” may occur by administration of one dose of a therapeutic agent or therapeutic agents, may occur by administration of one dose of a therapeutic agent or therapeutic agents, or may occur upon administration of a series of doses of a therapeutic agent or therapeutic agents. “Treating” or “treatment” does not require complete alleviation of signs or symptoms, and does not require a cure. “Treatment” can also refer to clinical intervention, such as administering one or more therapeutic agents to an individual, designed to alter the natural course of the individual being treated (i.c., to alter the course of the individual that would occur in absence of the clinical intervention).

The term “antimicrobial peptide” or “AMP” refers to a peptide that exerts an antimicrobial effect against one or more microbes.

The term “identity” between a peptide sequence and a reference sequence is the percentage of amino acid residues in the peptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides compositions comprising novel molecules with antibacterial properties and related methods for addressing the growing antibiotic resistance. In one aspect, the present disclosure provides a composition comprising a synthetic antimicrobial peptide (AMP). In some embodiments, the AMP exhibits potent antibacterial and antibiofilm efficacy against both gram-positive and gram-negative bacterial stains (c.g., MRSA and E. coli), reduced or minimal cytotoxicity and genotoxicity towards tissues and cells of the subject treated with the AMP (e.g., human fibroblasts), improved stability and targeted functionality, and/or capability of bypassing common bacterial resistance mechanisms. In some embodiments, the AMP selectively targets the bacterial wall thus providing a specific action on the pathogen. In another aspect, the present disclosure provides methods for treating various diseases such as infection-related diseases or conditions.

### Antimicrobial Peptides

In some aspects, the present disclosure provides AMPs. In some embodiments, the AMPs have potent antibacterial and/or antibiofilm activities, anti-inflammatory activity, and/or pro-apoptotic activity.

In some embodiments, the AMP comprises the amino acid sequence of AFCGGRCRGFRRRLFCTKAC (SEQ ID NO:1). In some embodiments, the AMP consists of the amino acid sequence of SEQ ID NO:1.

In some embodiments, the AMP comprises an amino acid sequence that has at least 80%, at least 85%, at least 90%, or at least 95% identity with SEQ ID NO:1. In some embodiments, the AMP consists of an amino acid sequence that has at least 80%, at least 85%, at least 90%, or at least 95% identity with SEQ ID NO:1.

In some embodiments, the AMP comprises the amino acid sequence of SEQ ID NO:1 with a substitution of at least one amino acid residue. For examples, the substitution may be of 1, 2, 3, 4, 5, 6, 7, 8, 9, 10, or more amino acid residues. In some embodiments, the substitution is a conservative amino acid substitution. For example, the substitution may be the replacement of one hydrophilic amino acid with another hydrophilic amino acid, the replacement of one hydrophobic amino acid with another hydrophobic amino acid, the replacement of an L-amino acid with a D-amino acid of the same identity, and/or the replacement of a D-amino acid with a L-amino acid of the same identity. In some embodiments, conservative amino acid substitutions are ones in which the amino acid residue is replaced with an amino acid residue having a similar side chain. Families of amino acid residues having similar side chains have been defined in the art. These families include: amino acids with acidic side chains (e.g., aspartate and glutamate), amino acids with basic side chains (e.g., lysine, arginine, and histidine), non-polar amino acids (e.g., alanine, valine, leucine, isoleucine, proline, phenylalanine, methionine, and tryptophan), uncharged polar amino acids (e.g., glycine, asparagine, glutamine, cysteine, serine, threonine and tyrosine), hydrophilic amino acids (e.g., arginine, asparagine, aspartate, glutamine, glutamate, histidine, lysine, serine, and threonine), hydrophobic amino acids (e.g., alanine, cysteine, isoleucine, leucine, methionine, phenylalanine, proline, tryptophan, tyrosine, and valine). Other families of amino acids include: aliphatic-hydroxy amino acids (e.g., serine and threonine), amide family (c.g., asparagine and glutamine), alphatic family (e.g., alanine, valine, leucine and isoleucine), aromatic family (c.g., phenylalanine, tryptophan, and tyrosine).

In some embodiments, the AMP comprises the amino acid sequence of SEQ ID NO:1 with a truncation of at least one amino acid residue (c.g., 1, 2, 3, 4, 5, or more) at the N-terminus. Alternatively, or additionally, in some embodiments, the AMP comprises the amino acid sequence of SEQ ID NO:1 with a truncation of at least one amino acid residue (c.g., 1, 2, 3, 4, 5, or more) at the C-terminus.

In some aspects, the present disclosure provides a composition comprising a polynucleotide comprising a coding sequence for SEQ ID NO:1. As used herein, a polynucleotide may be DNA, RNA, or a hybrid thereof. The polynucleotide may comprise natural nucleotides (such as A, T/U, C, and G), modified nucleotides, analogs of natural nucleotides, such as labeled nucleotides, or any combination thereof. In some embodiments, the polynucleotide may comprise one or more regulatory elements (or sequences encoding thereof), such as transcription control sequences, e.g., sequences which control the initiation, elongation and termination of transcription.

In some embodiments, the AMP can be produced using molecular and cell biology methods well known in the art, e.g., as described in Molecular Cloning: A Laboratory Manual, 4th edition (2012) (Green and Sambrook), PCT/US2013/030042, PCT/US2017/055596, PCT/US2019/051093, PCT/US2021/028254, and PCT/US2021/030277, cach of which is incorporated by reference in its entirety.

In some embodiments, the AMP can be produced by chemical synthesis. For example, the AMP may be synthesized using Liquid phase peptide synthesis (LPPS), or solid phase peptide synthesis (SPPS). Further examples of peptide synthesis methods include those described in Anderson G. W. and McGregor A. C. (1957) T-butyloxycarbonylamino acids and their use in peptide synthesis. Journal of the American Chemical Society. 79, 6180-3; Carpino L. A. (1957) Oxidative reactions of hydrazines. Iv. Elimination of nitrogen from 1, 1-disubstituted-2-arenesulfonhydrazidesl-4. Journal of the American Chemical Society. 79, 4427-31; Mckay F. C. and Albertson N. F. (1957) New amine-masking groups for peptide synthesis. Journal of the American Chemical Society. 79, 4686-90; Merrifield R. B. (1963) Solid phase peptide synthesis. I. The synthesis of a tetrapeptide. Journal of the American Chemical Society. 85, 2149-54; Carpino L. A. and Han G. Y. (1972) 9-fhiorenyhnethoxycarbonyl amino-protecting group. The Journal of Organic Chemistry. 37, 3404-9; and A Lloyd-Williams P. et al. (1997) Chemical approaches to the synthesis of peptides and proteins. Boca Raton: CRC Press. 278; U.S. Pat. Nos. 3,714,140, 4,411,994, 7,785,832, 8,314,208, 10,442,834, and US2005/0165215, each of which is incorporated by reference in its entirety.

### Pharmaceutical Compositions and Methods of Treatment

In some aspects, the present disclosure provides a pharmaceutical composition comprising a therapeutically effective amount of an AMP disclosed herein and one or more pharmaceutically acceptable carriers.

In some embodiments, the pharmaceutical composition is provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or acrosols.

In some embodiments, the pharmaceutical composition comprises one or more solvents. Examples of solvents include water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

In some embodiments, the pharmaceutical composition comprises one or more buffering agents. Examples of buffering agents include citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

In some embodiments, the pharmaceutical composition is a topical formulation, e.g., suitable for dermal application. In some examples, the pharmaceutical composition may comprise suitable excipients for topical application, e.g., suitable solvents, buffering agents, preservatives, humectants, chelating agents, antioxidants, stabilizers, emulsifying agents, suspending agents, gel-forming agents, ointment bases, penetration enhancers, and skin protective agents. Examples of solvents include water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof. Examples of buffering agents include citric acid, acetic acid, tartaric acid, lactic acid, hydrogenphosphoric acid, diethyl amine etc. Examples of preservatives include parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives. Examples of humectants include glycerin, propylene glycol, sorbitol, lactic acid, urea, and mixtures thereof. Examples of antioxidants include butylated hydroxy anisole (BHA), ascorbic acid and derivatives thereof, tocopherol and derivatives thereof, cysteine, and mixtures thereof. Examples of emulsifying agents include gums, e.g. naturally occurring gums such as gum acacia or gum tragacanth; phosphatides, c.g. naturally occurring phosphatides such as soybean lecithin, sorbitan monooleate derivatives: wool fats; wool alcohols; sorbitan esters; monoglycerides; fatty alcohols; fatty acid esters (e.g. triglycerides of fatty acids); and mixtures thereof. Examples of suspending agents include celluloses and cellulose derivatives such as, e.g., carboxymethyl cellulose, hydroxyethylcellulose, hydroxypropylcellulose, hydroxypropylmethylcellulose, carrageenan, acacia gum, arabic gum, tragacanth, and mixtures thereof. Examples of gel bases (e.g., viscosity-increasing agents or components which are able to take up exudate from a wound) include liquid paraffin, polyethylene, fatty oils, colloidal silica or aluminum, zinc soaps, glycerol, propylene glycol, tragacanth, carboxyvinyl polymers, magnesium-aluminum silicates, Carbopol, hydrophilic polymers such as, c.g. starch or cellulose derivatives such as, e.g., carboxymethylcellulose, hydroxyethylcellulose and other cellulose derivatives, water-swellable hydrocolloids, carrageenans, hyaluronates (c.g. hyaluronate gel optionally containing sodium chloride), and alginates including propylene glycol alginate. Examples of ointment bases include beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween). Examples of hydrophobic or water-emulsifying ointment bases include paraffins, vegetable oils, animal fats, synthetic glycerides, waxes, lanolin, and liquid polyalkylsiloxanes. Examples of hydrophilic ointment bases include solid macrogols (polyethylene glycols). Other examples of ointment bases include triethanolamine soaps, sulphated fatty alcohol and polysorbates. Examples of ointment base include anhydrous absorption base (c.g., Hydrophilic petrolatum, Lanolin, Aquaphor, Aquabase, Polysorb), water-in-oil emulsion base (c.g., Hydrous lanolin, Cold cream, Eucerin, Hydrocream, Nivea), and oil-in-water emulsion base (c.g., Hydrophilic ointment, vanishing cream, Dermabase, Velvachol), and Aquaphor. Examples of other excipients include polymers such as carmellose, sodium carmellose, hydroxypropylmethylcellulose, hydroxyethyl cellulose, hydroxypropyl cellulose, pectin, xanthan gum, locust bean gum, acacia gum, gelatin, carbomer, emulsifiers like vitamin E, glyceryl stearates, cetearyl glucoside, collagen, carrageenan, hyaluronates and alginates and chitosans.

In some embodiments, in the pharmaceutical composition, the AMP is formulated in an aqueous liquid dispersion, self-emulsifying dispersion, solid solution, liposomal dispersion, aerosol, solid dosage form, powder, immediate release formulation, controlled release formulation, fast melt formulation, tablet, capsule, pill, delayed release formulation, extended release formulation, pulsatile release formulation, multiparticulate formulation (c.g., nanoparticle formulation), and mixed immediate and controlled release formulation. In some examples, the formulation is a nanoparticle formulation (e.g., the AMP is formulated in a nanoparticle formulation). The nanoparticle may be or comprise cMAP, cyclodextrin, lipids, solid lipid nanoparticle, polymeric nanoparticle, self-emulsifying nanoparticle, liposomes, microemulsion, micellar solution, paramagnetic nanoparticle, superparamagnetic nanoparticle, metal nanoparticle, fullerene-like material, inorganic nanotube, dendrimer, nanofibers, nanohorn, nano-onion, nanorod, nanorope, quantum dots, or any combination thereof. In some examples, the nanoparticle is a nanofiber. In some examples, the nanofiber is a graphene nanofiber. Nanofibers are polymeric and/or composite fibers that have a nanoscale diameter. Nanofibers may have porous structures and an extensive area.

In some aspects, the present disclosure provides methods of treating a disease or condition by administering the AMP or pharmaceutical composition to a subject. The disease or condition may be an infection-related disease or condition, e.g., bacterial infection, wound, skin disorders, inflammatory diseases, aging, cancer, etc.

In some embodiments, the bacterial infection is an infection by a gram-negative bacterium. Examples of gram-negative bacteria include Enterobacteriaceae (e.g., Escherichia, c.g., E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (c.g., Pseudomonas, c.g., P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (c.g., Treponema and Borrelia), Leptospiraceae, Campylobacter, Helicobacter, Spirillum, Streptobacillus, Bacteroidaceae (c.g., Bacteroides, Fusobacterium, Prevotella, Porphyromonas), Acinetobacter, c.g., A. baumannii. Examples of gram-positive bacteria include Listeria monocytogenes, Staphylococcus aureus (c.g., Methicillin-resistant Staphylococcus aureus, MRSA), Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium tuberculosis, Corynebacterium diphteriae, Mycoplasma pneumoniae, and Actinomyces.

In some embodiments, the infection is an infection of the skin, soft tissues, the respiratory system, the lung, the digestive tract, the eye, the car, the teeth, the nasopharynx, the mouth, the bones, the vagina, of wounds of bacteracmia and/or endocarditis caused by Gram-negative and/or Gram-positive bacteria.

In some embodiments, the disease is an auto-inflammatory skin disease. Autoinflammatory diseases are a group of diseases characterized by recurrent episodes of systemic inflammation, and/or with an increased production of inflammatory cytokines that can occur without detectable autoantibodies or auto-reactive T cells. An auto-inflammatory skin disease may be the skin manifestation of an autoinflammatory disease. An auto-inflammatory skin disease may be urticarial exanthema, dermo-hypodermitis, neutrophilic dermatosis, granulomatous dermatitis, or bipolar aphthosis. Examples of auto-inflammatory skin diseases include autoinflamatory urticarial dermatosis such as CAPS; autoinflammatory neutrophilic dermatosis such as pyogenic sterile arthritis, pyoderma gangrenosm, and acne (PAPA) syndrome, pustular psoriasis, palmoplantar pustulosis, subcorneal pustulosis, hidradenitis suppurativa, severe acne, folliculitis decalvans, crosive pustular dermatosis of the scalp, neutrophilic urticarial dermatosis, erythema clevatum diutinum, and Behcet's disease; autoinflammatory granulomatosis such as Blau syndrome; autoinflammatory chilblain lupus such as Aicardi-Goutieres syndrome (AGS); autoinflammatory lipoatrophy such as Nakajo-Nishimura syndrome (NNS); autoinflammatory angioedema such as hereditary angioedema (HAE); and probable autoinflammatory bullous disease such as granular C3 dermatosis (GCD). In one example, the auto-inflammatory skin discase is hidradenitis suppurativa.

In some embodiments, the route of administration of the AMP or pharmaceutical composition may be oral, topical, nasopharyngeal, parenteral, inhalational, intravenous, intramuscular, intrathecal, intraspinal, endobronchial, intrapulmonary, intraosscous, intracardial, intraarticular, rectal, vaginal, or any other route of administration. In some embodiments, the AMP or pharmaceutical composition is administered topically. Topical administration may involve administration on a rash, wound, lesion, abscess, sore, blister, pimple, lump, wart, boil, and the like.

In some embodiments, the AMP and pharmaceutical composition can be used for wound management and/or skin condition treatments. They may be used for managing acute and chronic wounds. In some embodiments, the AMP and pharmaceutical composition can be used to address critical needs in hospitals, outpatient clinics, and healthcare systems, c.g., by promoting faster healing, reducing infection risks, and enhancing tissue regeneration. In some embodiments, the applications of the AMP and pharmaceutical composition include dermatological practices, where they are used for treating various skin disorders, improve skin integrity, and manage inflammatory conditions. In some embodiments, using the AMP and pharmaceutical composition in health care systems allows more efficient patient care, reduced hospital stays, and cost savings associated with wound management. The AMP and pharmaceutical composition may be used in cosmetics and personal care products, c.g., anti-ageing solutions, skin rejuvenation therapies, and treatments for conditions such as acne.

In some embodiments, the AMP and pharmaceutical composition can enhance skin health and appearance. For example, the AMP and pharmaceutical composition can be used for skin treatment in spas, beauty salons, and cosmetic clinics.

### EXAMPLES

### Example 1—AMP with Potent Antibacterial and Antibiofilm Capacities Against MRSA and E. coli Clinical Isolates Through in Silico Design

This example shows the design and evaluation of a synthetic antimicrobial peptide (AMP). The AMP was designed using computational search and further engineering through molecular docketing and dynamics. The AMP underwent rigorous testing for antibacterial and antibiofilm activities against exemplary bacterial strains Methicillin-Resistant Staphylococcus Aureus (MRSA) and Escherichia coli (E. coli), representing gram-positive and gram-negative bacteria, respectively. Subsequently, the safety profile of the AMP was assessed in vitro using human fibroblast cells. In general, the selected AMP demonstrated robust antibacterial and antibiofilm efficacy against MRSA and E. coli, with an added assurance of non-cytotoxicity and genotoxicity towards human fibroblasts.

### AMP Synthesis and Characterization

The study was initiated by selecting AMP sequences from publicly available repositories such as the Collection of Antimicrobial peptides (CAMP), CAMPSign, and ClassAMP. These AMP sequences served as probes for identifying homologous peptides of different Eukaryotic species, provided their genomes or transcriptomes were accessible in online repositories. Subsequently, AMP analogs were designed based on the mature peptide sequences obtained in the previous step. The design criteria included targeting peptides of approximately 10-34 amino acids in size, possessing a cationic surface charge, and showing potential for antimicrobial and therapeutic activity.

Next, three-dimensional structure analysis of these AMPs was performed. To obtain their three-dimensional models, the ROSETTA package for ab initio modeling was used (as described in Rohl C A et al., Protein structure prediction using Rosetta. Methods Enzymol. 2004, 383, 66-93). The quality of each model generated through these methods was then rigorously assessed using tools such as the Ramachandran graph (as described in Lovell S C et al., Structure validation by Calpha geometry: phi,psi and Cbeta deviation. Proteins 2003, 50, 437-450) to evaluate stereochemical characteristics and the Z-score with ProSa II to gauge the folding quality (as described in Wiederstein M et al., ProSA-web: interactive web service for the recognition of errors in three-dimensional structures of proteins. Nucleic Acids Res 2007, 35, W407-410).

The investigation also encompassed molecular docking and dynamics (MD) simulations. The ClusPro server (as described in Kozakov D et al., The ClusPro web server for protein-protein docking. Nat Protoc 2017, 12, 255-278) was harnessed for global molecular docking, maintaining the main receptor chain as rigid while allowing the peptide to exhibit greater flexibility in searching for the lowest energy binding region. This process was repeated extensively, resulting in an energy cluster from which the program ranked the top ten binding models between the protein and the peptide. To evaluate the stability of these models, molecular dynamics simulations were conducted before and after the docking process using GROMACs 2019.4 (as described in Abraham M J et al., GROMACS: High performance molecular simulations through multi-level parallelism from laptops to supercomputers. SoftwareX 2015, 1-2, 19-25). The simulations employed the GROMOS96 force field 53a6 (as described in Oostenbrink C et al., Validation of the 53A6 GROMOS force field. Eur Biophys J 2005, 34, 273-284) and the SPC water model (as described in Berendsen HJC et al., Interaction Models for Water in Relation to Protein Hydration. In: Pullman, B. (eds) Intermolecular Forces. The Jerusalem Symposia on Quantum Chemistry and Biochemistry. Intermolecular Forces 1981, 14). The system was solvated with NaCl ions to reach a physiological concentration of 0.15M, followed by energy minimization. Dynamics were carried out at a temperature of 300 K and a constant pressure of 1 atm for 100 ns. Following this time frame, the models were evaluated and compared based on their three-dimensional structure, secondary structure, and system energy.

Finally, NovoPro Bioscience Inc. (Shanghai, China) commercially synthesized the peptides selected through the bioinformatics pipeline. NovoPro Bioscience conducted assessments of peptide purity using reverse-phase high-performance liquid chromatography (HPLC) and subjected the peptides to mass spectrometry for quality control and molecular weight confirmation.

The sequence of the designed peptide is AFCGGRCRGFRRRLFCTKAC (SEQ ID NO:1).

Growth of MRSA and E. coli Strains

MRSA and E. coli strains were acquired from the microbiology bacterial culture stock at the Biomedical Research Centre (BRC), Qatar University. MRSA (ATCC-33591) and E. coli (ATCC-29522) strains were cultivated overnight on nutrient agar (Remel, ThermoFisher Scientific, Lenexa, KS, USA), followed by a 24 hour period of incubation at 37° C. and adjustment to 0.5 MacFarland Standard, which was measured using DensiCHEK PLUS (bioMérieux, France).

### AMP Activity

Minimal Inhibitory Concentration (MIC) of AMP MRSA and E. coli was determined as follows. Six serial dilutions of AMP starting from 2000 μg/ml of 160 μL Mueller Hinton broth (MHB, Lioflchem, Roseto degli Abruzzi, Italy) were transferred to the wells (100 μl each) in a 96-well flat-bottom plate (Microtesttm 96 tissue culture plate, Franklin Lakes, NJ, USA) to get concentrations of 2000, 1000, 500, 250, 125, 62.5, 31.25, 15.6, and 7.8 μg/mL. Then, 10 μL of 0.5 MacFarland for each type (MSRA and/or E. coli) of the measured strain was added to each well. To assure sterilization, the 96-well plate contained wells with MHB control, MHB with AMP treatment control, and controls for the bacterial strain mixed with MHB to guarantee bacterial growth. 96-well plates were then sealed tightly with a parafilm and placed in an aerobic shaker (Innova 44 Incubator Shaker) for 24 hours at 37° C. at a speed of 100 rpm. After this, the MIC of the treatment that effectively eliminated bacterial growth was determined with the naked eye following previously established procedure as described in Eltai N O et al., Antibacterial and Antibiofilm Activity of Mercaptophenol Functionalized-Gold Nanorods Against a Clinical Isolate of Methicillin-Resistant Staphylococcus aureus. J Inorg Organomet Polym 2022, 2527-2537; and Rodríguez-Melcón C et al., Minimum Inhibitory Concentration (MIC) and Minimum Bactericidal Concentration (MBC) for Twelve Antimicrobials (Biocides and Antibiotics) in Eight Strains of. Biology (Basel) 2021, 11.

To determine the minimum biocidal concentration (MPC) for the AMP, 20 μL of a ten-fold serial dilution of the MIC with AMP was spread onto fresh nutrient agar media and incubated at 37° C. for around 18 hrs. Bacterial colonies were counted and compared to those grown on control nutrient agar, which had 20 μL of a solution composed only of bacteria and MHB.

Effect of AMP on MRSA Biofilm and E. coli Biofilm
MRSA Biofilm and E. coli Biofilm Culture

The first step in creating the biofilm was the selection of borosilicate glass beads 3-4 mm in diameter (ISOLAB Laborgeräte GmbH, Singapore). Then, the selected beads were soaked and cleaned with a soap solution; after that, they were washed with ddH2O, soaked in 80% ethanol for 24 hrs, and then washed properly using sterile water. Bacteria were then grown on these beads to create the biofilm by taking 20 μL of 0.5 McFarland from the bacterial strains (either MRSA or E. Coli), adding it to 200 μL of nutrient broth in 96-well flat-bottom plates, and placing one bead in cach well. 96-well plates were then sealed tightly with a parafilm and placed in an acrobic shaker (Innova 44 Incubator Shaker) for 72 hours at 37° C. at a speed of 100 rpm. Every 24 hours, 200 μL of fresh media was added to replace the consumed media. The beads were then placed in an Eppendorf tube containing 200 μL of nutrient broth and vortexed for 1 min to loosen any attached bacteria. This step was repeated after 24, 48, and 72 hours to observe the density of the biofilm formed on the beads. Then, 100 μL of the solution was serially diluted over ten-fold serial dilutions, and 20 μL of the serial dilution was spread onto fresh nutrient agar media and incubated at 37° C. for 18 hours. Biofilm colonies were then counted. Generated biofilms were used to investigate the selected AMP's ability to inhibit the MRSA and E. coli biofilms.

AMP Activity Against MRSA and E. coli Biofilm

The antibiofilm activity of AMP on 72 hours biofilm cultures was tested by treating the biofilm beads with 2000, 1000, 500, 250, 125, and 62.5 μg/mL concentrations of the selected AMP in 96-well plates. Plates were then tightly sealed with a parafilm and placed in an acrobic shaker (Innova 44 Incubator Shaker) for 72 hours at 37° C. at a speed of 100 rpm. The antibiofilm activity of the AMP was determined after observing the wells that contained a transparent solution with the naked eye. Subsequently, the antibiofilm activity of the selected AMP was determined using SEM, as described below.

Imaging of MRSA, E. coli, MRSA Biofilm, and E. coli Biofilm Using Scanning Electron Microscopy (SEM)

Beads containing bacteria and biofilms were frecze-dried, fixed to a stub with sticky carbon tape, and then completely spray-sprayed with a 12-nm layer of gold. SEM (ZEISS 1530 Gemini, Carl Zeiss Microscopy GmbH, Germany) was used to examine and photograph the surface of the beads.

### In Vitro Cytotoxicity and Genotoxicity Assessment of the AMP

The cytotoxicity and genotoxicity in vitro effect of the AMP was investigated. Human Fibroblasts (ATCC PCS201010, ThermoFischer Scientific, USA) and cells were seeded at a seeding density of 10,000 Cells/well in 96-well plates using Dulbecco's Modified Eagle Medium (DMEM, Gibco, USA). Media was supplemented with 10% Fetal bovine serum (FBS, Gibco, USA), and penicillin streptomycin (Gibco, USA). Cells were incubated overnight (16-18 hrs) at humidified conditions (5% CO₂, 37° C.). Adherent cells were then treated with different concentrations of the AMP (7.8125, 15.625, 62.5, 125, 250, 500, 1000, 2000 μg/ml), and cells were then incubated at humidified conditions for 24 hrs. Then conditioned media was collected at different time points 0, 0.5, 1, 3, 6, 24 hours) and used for measuring Lactate Dehydrogenase (LDH, Thermofischer, USA) following manufacturers' instructions. Adherent cells were used to measure cell viability using AlamarBlue (Invitrogen, USA) following manufacturers' instructions at the following time points 0, 0.5, 1, 3, 6, and 24 hours.

The genotoxicity effect of the selected AMP was also investigated. In this part, human fibroblast cells were cultured in 24-well plates at a seeding density of 100,000 cells/well; cells were then allowed to adhere and proliferate overnight (16-18 hours). The following day, cells were treated with the AMP (1000 and 2000 μg/ml; negative and positive controls were included in this part, and cells were treated with DMEM and H₂O₂, respectively. After which, cells were incubated at humidified conditions for 24 hours. Following that, the genotoxicity effect of the AMP was measured using oxiselect comet assay (Abcam, UK) and following the manufacturer's instructions. Images were taken using OLYMPUS BX62, at magnification 40×, attached to an OLYMPUS DP73 digital camera. Image analysis was done using the ImageJ application (OpenComet plugin).

### Statistical Analysis

Obtained results were analyzed and presented as mean±S.E.M for n experiments with well-defined figure legends. All statistical tests were performed using GraphPad Prism v5. Statistical analysis for AMP effect was determined using two-way ANOVA or one-way ANOVA followed by Bonferroni Post-tests. Statistical significance was noted at *p<0.05.

### Results

Antibacterial Activity of AMP Against MRSA and E. coli

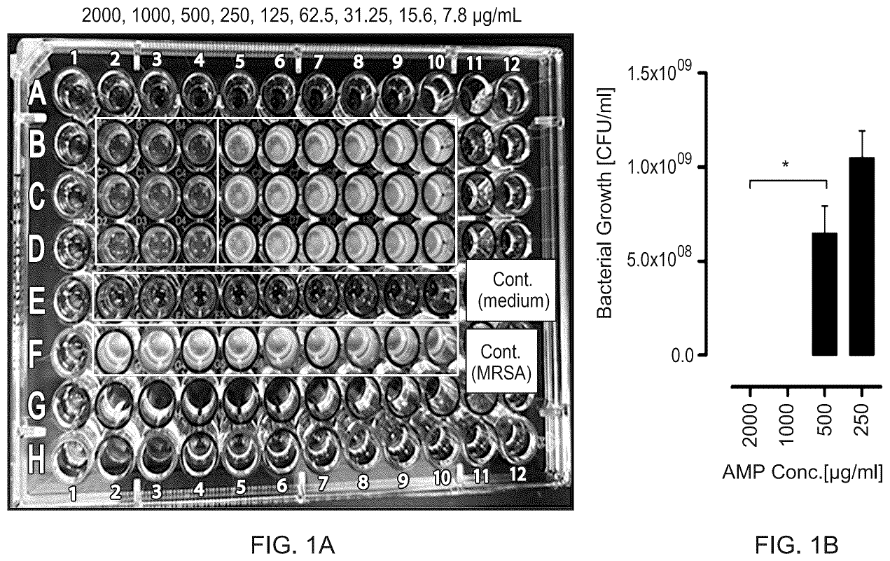

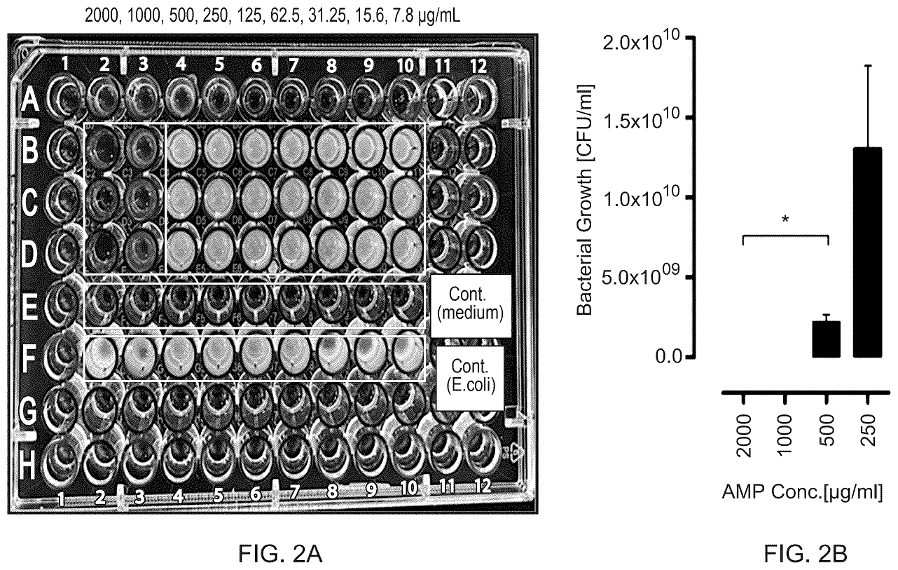

The antibacterial activity of the selected AMP was evaluated using MRSA and E. coli by exposing the bacteria to different AMP concentrations for 24 hours. Results showed that the concentrations of the peptide 2000, 1000 μg/ml, demonstrated biocidal effect to the clinical MRSA and E. coli strain used in this study, while there was a reduction in bacterial growth at 500 μg/ml AMP concentration with a log reduction of 3 for the MRSA and 2 for the E. coli (FIGS. 1A and 2A). In turn causing a concentration-dependent change in the color of the solution compared to the other AMP concentrations (250, 125, 62.5, 31.25, 15.6, and 7.8 μg/ml). Moreover, the findings showcased that the minimum inhibitory (MIC) and MBC of the AMP using MRSA and E. coli was 500 and 1000 μg/ml, respectively (FIGS. 1B and 2B), thus, demonstrating the efficacy of AMP as an antibacterial agent capable of inhibiting both MRSA and E. coli strains.

### Bacteria Biofilm Growth

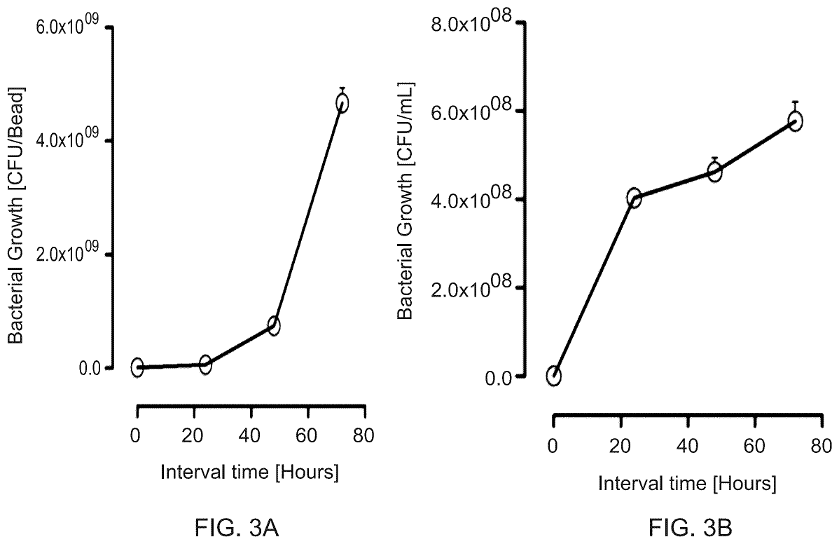

The MRSA and E. coli biofilms were developed on the beads' surface. The biofilm growth was monitored and reported by counting the number of the biofilms formed on the beads at various time intervals, including 0 hour, 24 hours, 48 hours, and 72 hours. MRSA biofilm showed exponential growth and continued to increase over time (FIG. 3A), while E. coli biofilm was increased up to 24 hours, after which it plateaus (FIG. 3B).

Antibiofilm Activity of AMP Against MRSA and E. coli Biofilms

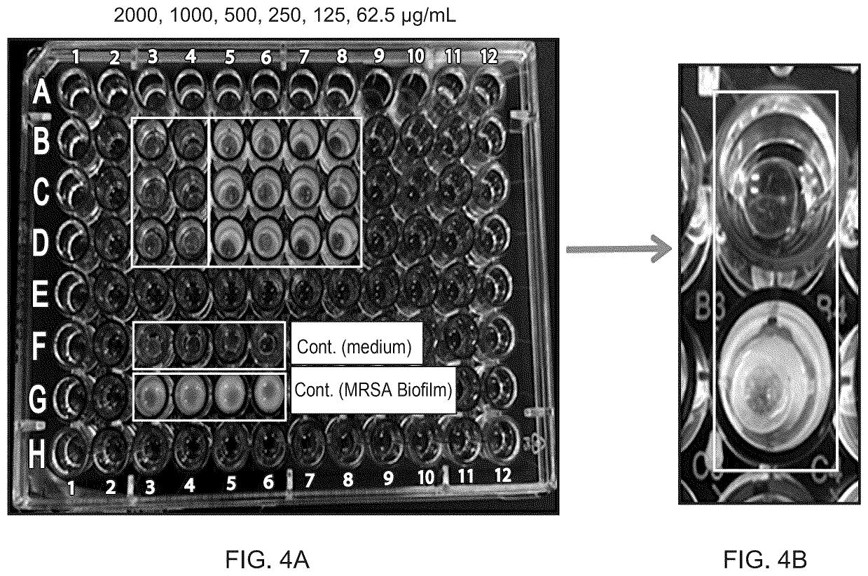

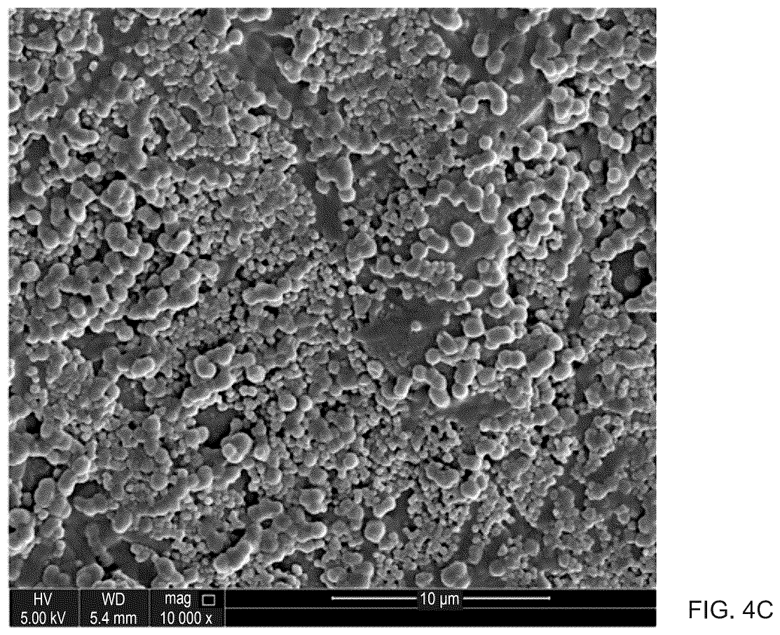

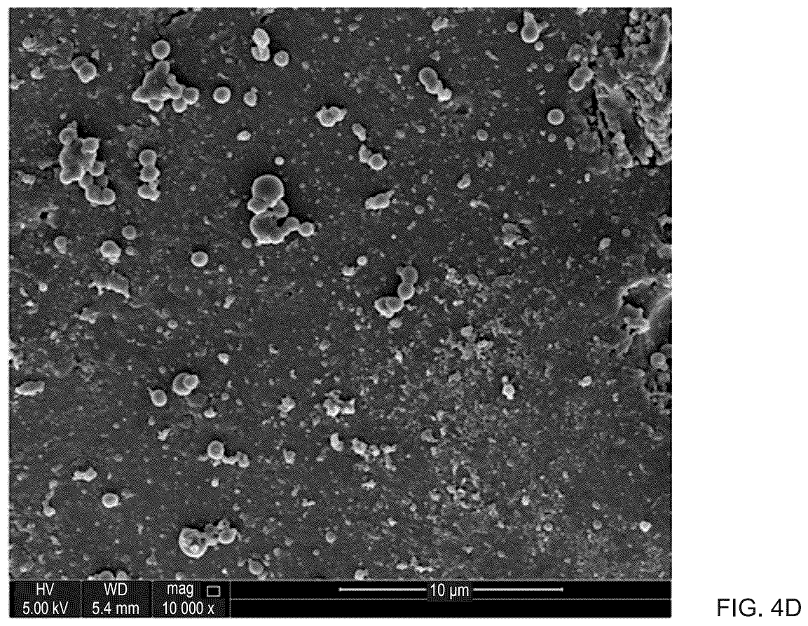

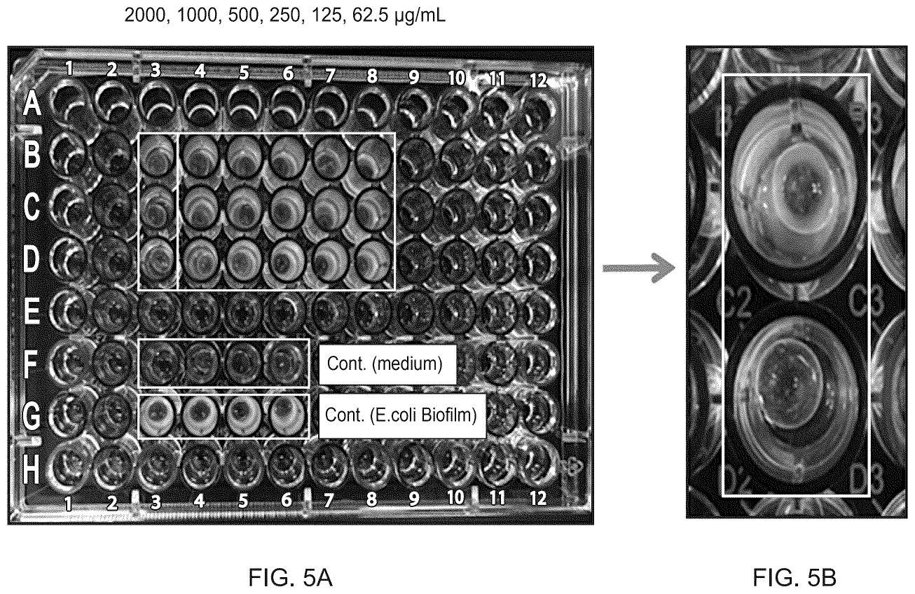

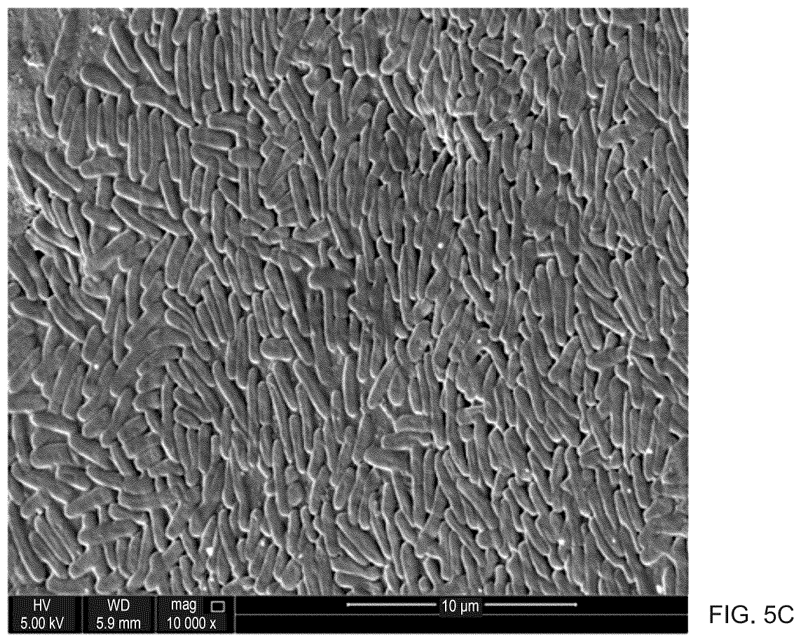

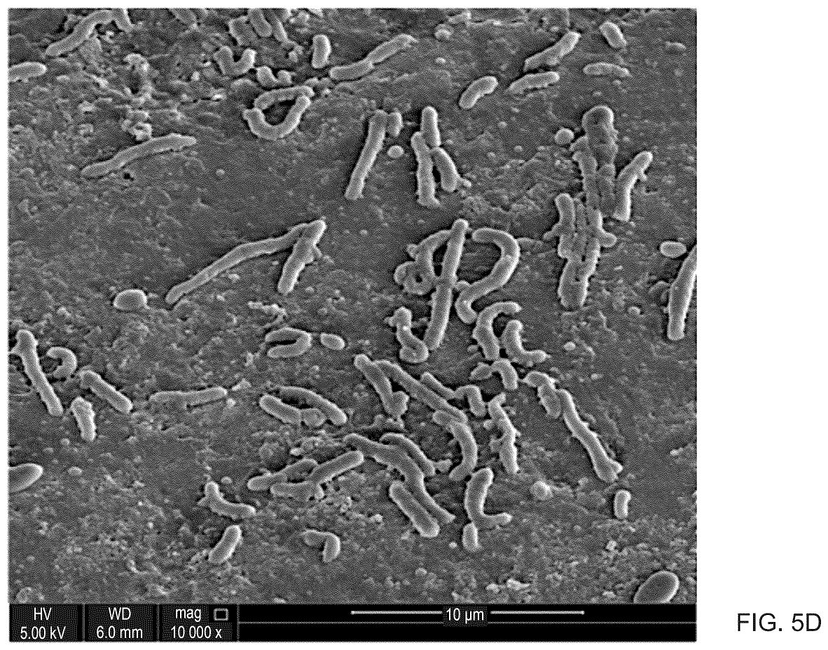

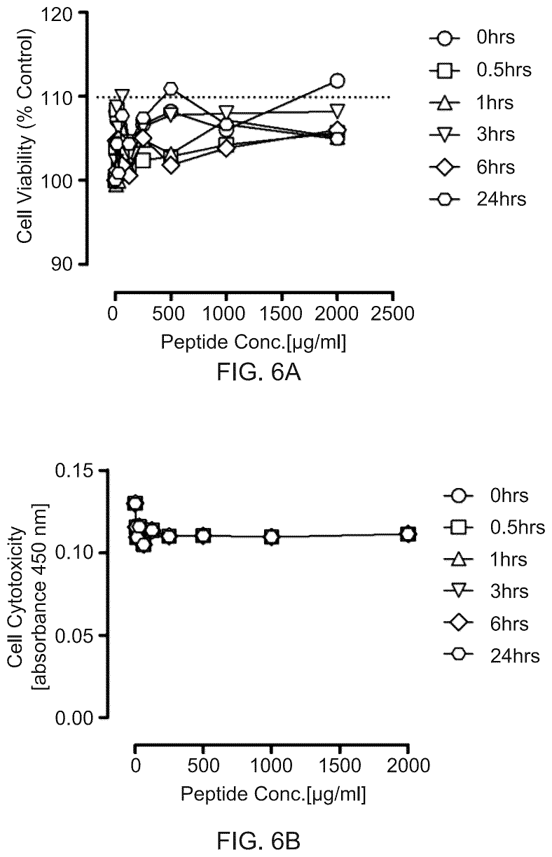

The effect of different concentrations of the AMP on the MRSA and E. coli biofilms was investigated, with results demonstrating that the first two concentrations of the peptide (2000,1000 μg/ml) were able to eliminate the MRSA biofilm (FIGS. 4A-4D). On the other hand, only the highest concentration, 2000 μg/ml, could inhibit the E. coli biofilm (FIGS. 5A-5D). Comparably, the change in the media color in the wells containing the beads was observed and paralleled to the remaining concentrations (FIGS. 4A and 5A). Furthermore, results showed that the MIC/MBC for the AMP in the MRSA biofilm is 1000 μg/mL (FIG. 4A). With the highest dose of the treatment (2000 μg/ml), totally inhibiting the MRSA biofilm growth (FIG. 4B). On the contrary, the MIC/MBC for E. coli biofilm was 2000 μg/mL (FIG. 6A), with the highest dose of the AMP having the efficacy to inhibit the E. coli biofilm growth (FIG. 6B).

The SEM images of the surface of glass beads show the difference in the density of the control group of the MRSA biofilm growth compared to the density of the MRSA and E. coli biofilms treated with the highest dose of the treatment (2000 μg/ml) (FIGS. 4C, 4D, 5C, and 5D). The findings show the effectiveness of the AMP in inhibiting the growth of the MRSA and E. coli biofilm used in the study.

### In Vitro Assessment of the AMP

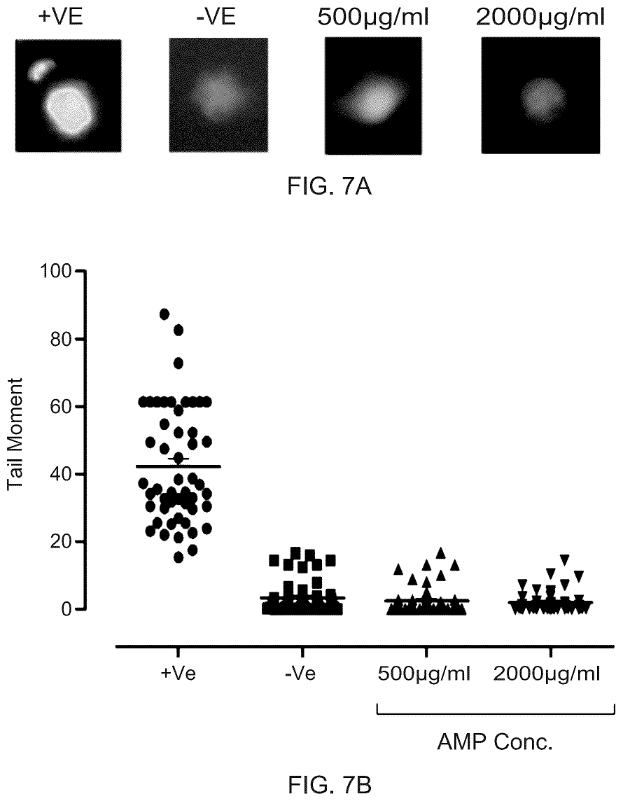

Results showed that the tested AMP did not affect the cells' viability at all the tested concentrations and time points (FIG. 6A). Moreover, no cellular cytotoxicity was detected using different AMP concentrations (FIG. 6B). Finally, genotoxicity results showed that there was no significant effect caused by the two AMP concentrations showing antimicrobial activity against MRSA and E. coli (FIGS. 7A and 7B).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising a peptide comprising a sequence that has at least 85% identity with the amino acid sequence set forth in SEQ ID NO:1.

2. The composition of claim 1, wherein the peptide has at least 90% identity with the amino acid sequence set forth in SEQ ID NO:1.

3. The composition of claim 1, wherein the peptide has at least 95% identity with the amino acid sequence set forth in SEQ ID NO:1.

4. The composition of claim 1, wherein the peptide comprises the amino acid sequence set forth in SEQ ID NO:1.

5. A pharmaceutical composition comprising a therapeutically effective amount of the peptide of claim 1 and a pharmaceutically acceptable carrier.

6. The pharmaceutical composition of claim 5, wherein the peptide is formulated in a nanoparticle.

7. The pharmaceutical composition of claim 6, wherein the nanoparticle comprises nanofibers.

8. The pharmaceutical composition of claim 7, wherein the nanofibers comprise graphene nanofibers.

9. A method of treating or preventing a disease comprising administering the pharmaceutical composition of claim 5 to a subject in need thereof.

10. The method of claim 9, wherein the disease is a bacterial infection.

11. The method of claim 10, wherein the bacterial infection is an infection by a gram-positive bacterium.

12. The method of claim 11, wherein the gram-positive bacterium is Staphylococcus aureus.

13. The method of claim 12, wherein the Staphylococcus aureus is Methicillin-Resistant Staphylococcus aureus.

14. The method of claim 10, wherein the bacterial infection is an infection by a gram-negative bacterium.

15. The method of claim 14, wherein the gram-negative bacterium is Escherichia coli.

16. The method of claim 9, wherein the disease is skin infection.

17. The method of claim 9, wherein the disease is an auto-inflammatory skin disease.

18. The method of claim 17, wherein the auto-inflammatory skin disease is hidradenitis suppurativa.

19. The method of claim 9, wherein the disease is cancer.
